(19) United States
(12) Patent Application Publication
(10) Pub. No.: US 2010/0041697 A1
(43) Pub. Date: Feb. 18, 2010

(54) PNEUMONIA TREATMENT

(75) Inventors: Kit Chiu, South San Francisco, CA (US); Luke Lin, Singapore (SG); Hao-Chen Tan, Taipei (TW); Chi-Hsin Richard King, Holladay, UT (US); Ming-Chu Hsu, Glendora, CA (US)

Correspondence Address: OCCHIUTI ROHLICEK & TSAO, LLP, 10 FAWCETT STREET, CAMBRIDGE, MA, 02138, US

(73) Assignee: TaiGen Biotechnology Co., Ltd., Taipei (TW)

(21) Appl. No.: 12/495,982

(22) Filed: Jul. 1, 2009

Related U.S. Application Data
(60) Provisional application No. 61/077,599, filed on Jul. 2, 2008.

(51) Int. Cl. A61K 31/4709

(52) U.S. Cl. 514/312

(57) ABSTRACT

This invention relates to a method of treating pneumonia by orally administering to a subject in need thereof a quinolone compound of formula (I), shown in the disclosure, at a daily dose of 2-30 mg/kg.

### CROSS REFERENCE TO RELATED APPLICATIONS

This application claims priority to U.S. Provisional Application Ser. No. 61/077,599, filed Jul. 2, 2008. The content of the prior application is hereby incorporated by reference in its entirety.

### BACKGROUND OF THE INVENTION

Pneumonia, infection of the lung, is a leading cause of death among elderly people and terminal patients. Typical symptoms of pneumonia include cough, chest pain, fever, and difficulty in breathing.

Attempting to identify a patient's risk factors when he or she first comes to medical attention, clinicians have often classified pneumonia into two broad categories: community-acquired pneumonia (contracted outside hospitals and healthcare facilities) and hospital-acquired pneumonia (contracted within 48-72 hours of being admitted in a hospital). Recently, a third category has been introduced—healthcare-associate pneumonia—to include pneumonia contracted by a person living outside the hospital yet having been in close contact with the health care system.

Antibiotics can be used to treat pneumonia of all three categories. Recent efforts have been focused on developing more effective antibiotic drugs, ideally effective in treating drug-resistant bacterial pneumonia.

### SUMMARY

This invention relates to a method of treating pneumonia (e.g., community-acquired pneumonia) by orally administering to a subject a composition containing a quinolone compound of formula (I):

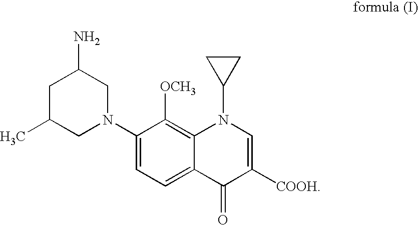

This method requires that the daily dose of the quinolone compound range from 2-30 mg/kg, for example, 3-16 mg/kg, 3-7 mg/kg, and 7-12 mg/kg.

The above-shown quinolone compound can be the compound itself as shown above, or its salt, prodrug, or solvate. A salt can be formed between an anion and a positively charged group on a compound. Suitable anions include chloride, bromide, iodide, sulfate, nitrate, phosphate, citrate, methanesulfonate, trifluoroacetate, acetate, malate, tosylate, tartrate, fumurate, glutamate, glucuronate, lactate, glutarate, and maleate. Likewise, a salt can also be formed between a cation and a negatively charged group on the compound. Suitable cations include sodium ion, potassium ion, magnesium ion, calcium ion, and an ammonium cation (e.g., tetramethylammonium ion). A prodrug can be ester and another pharmaceutically acceptable derivative, which, upon administration to a subject, is capable of the quinolone compound described above. A solvate refers to a complex formed between the quinolone compound and a pharmaceutically acceptable solvent. A pharmaceutically acceptable solvent can be water, ethanol, isopropanol, ethyl acetate, acetic acid, and ethanolamine. The compound used to practice this invention can therefore be, for example, the malate salt of the quinolone compound shown above and the hemihydrate of the malate salt.

The quinolone compound contains asymmetric centers. It can be in any form of stereoisomers. Two examples of isomeric compounds are:

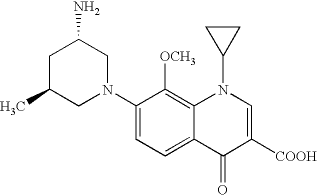

### (3S,5S)-7-[3-amino-5-methyl-piperidinyl]-1-cyclopropyl-1,4-dihydro-8-methoxy-4-oxo-3-quinolinecarboxylic acid

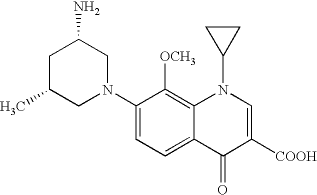

### (3 S,5R)-7-[3-amino-5-methyl-piperidinyl]-1-cyclopropyl-1,4-dihydro-8-methoxy-4-oxo-3-quinolinecarboxylic acid

Details of several embodiments of the invention are set forth in the description below. Other features, objects, and advantages of the invention will be apparent from the description, and also from the claims.

### DETAILED DESCRIPTION

The quinolone compound used to practice this invention can be synthesized by conventional methods. Example 1 below illustrates synthetic methods to prepare two isomeric compounds. Other isomers or forms, as recognized by those skilled in the art, can be synthesized by modified synthetic methods. Synthetic chemistry transformations and protecting group methodologies (protection and deprotection) useful in the synthesis are known in the art and include, for example, those described in R. Larock, Comprehensive Organic Transformations, VCH Publishers (1989); T. W. Greene and P. G. M. Wuts, Protective Groups in Organic Synthesis, 3^{rd} Ed., John Wiley and Sons (1999); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995) and subsequent editions thereof.

The compound thus synthesized can be further purified by flash column chromatography, high performance liquid chromatography, crystallization, or any other suitable methods.

To prepare the oral composition used in this method, one can mix the quinolone compound with one or more excipients at a predetermined ratio in any sequence. An excipient may be a binder, a disintegrant, a filler, a diluent, a glidant, a lubricant, and/or an antiadherent. See, e g., Sam, Drug Information Journal, 2000, Vol. 34, pp. 875-894. Mixing can be achieved by shaking, agitation, or swirling and is controlled to reconstitute the quinolone compound into the excipients (e.g., microcrystalline cellulose and magnesium stearate). At any stage of the preparation, sterilization, e.g., by an autoclave, may be applied. If desired, certain sweetening, flavoring, or coloring agents can be added.

The composition of this invention can be enclosed in capsule shells. The capsule shells can be formed of a material that is well recognized by one skilled in the art, for example, porcine collagen material (e.g., porcine collagen or gelatin), bovine collagen material, gelatin, gum arabic, pectin, poly(ethylene-co-maleic anhydride), poly(vinvlmethylether-co-maleic anhydride), carrageenan, and agar-agar.

The composition can be also compressed to form tablets. To enhance bioavailability, one may reduce the particle size of the compound to 10-50 microns before mixing it with excipients.

To practice the method of this invention, the above-described capsules or tablets are orally administered to a pneumonia patient in a controlled amount so as to ensure the desired daily dose, e.g., 2-30 mg/kg of the quinolone compound.

As used herein, the term “treating” or “treatment” refers to the administration of the quinolone compound to a subject, who has pneumonia, a symptom of the pneumonia, a disease or disorder secondary to the pneumonia, or a predisposition toward the pneumonia, with the purpose to cure, alleviate, relieve, remedy, or ameliorate the pneumonia, the symptom of, the disease or disorder secondary to, or the predisposition toward the pneumonia.

Pneumonia may result from infection with bacteria, including Streptococcus pneumoniae, Staphylococcus aureus, Haemophilus influenzae, Klebsiella pneumoniae, Escherichia coli, Pseudomonas aeruginosa, and Moraxella catarrhalis. These bacteria may be nonsusceptible to methicillin, vancomycin, or penicillin. The term “nonsusceptible” refers to resistance to a drug at the intermediate level up to the full level.

The term “daily dose” refers to the weight of the active agent administered to the subject based on one kilogram of the body weight of the subject each day during the treatment. When the active agent is a salt, prodrug, or solvate, the weight of the active agent use to calculate the daily dose is that of the quinolone compound itself (the molecular weight of which is 371), not that of the salt, prodrug, or solvate. For example, if a malate salt hemihydrate is administered to a subject having the body weight of 60 kg, the daily dose is calculated as follows:

$$daily dose = \frac{\begin{matrix}weight of quinolone compound \\ administered within one day\end{matrix}}{60 kg} \qquad$$

The capsules or tablets may be administered from 1 to 6 times, e.g., once, twice, or thrice, per day to reach the desired daily dose. The length of the treatment can be readily determined by a skilled person in the art based on pharmacokinetic study. For example, it may be 1-30 days, or 5-15 days, or 7-10 days.

Without further elaboration, it is believed that the above description has adequately enabled the present invention. The following examples are, therefore, to be construed as merely illustrative, and not limitative of the remainder of the disclosure in any way whatsoever. All of the publications cited herein are hereby incorporated by reference in their entirety.

### Example 1

Malate salt hemihyrdate of (3S,5S)-7-[3-amino-5-methyl-piperidinyl]-1-cyclopropyl-1,4-dihydro-8-methoxy-4-oxo-3-quinolinecarboxylic acid (Compound 1) and malate salt hemihydrate of (3S,5R)-7-[3-amino-5-methyl-piperidinyl]-1-cyclopropyl-1,4-dihydro-8-methoxy-4-oxo-3-quinolinecarboxylic acid (Compound 1′) were synthesized as follows:

### (A) Synthesis of (3S,5S)-(5-Methyl-piperidin-3-yl)-carbamic acid tert-butyl ester (Compound 9) and (3S,5R)-(5-Methyl-piperidin-3-yl)-carbamic acid tert-butyl ester (Compound 9′)

Compound 9 was synthesized as shown in Scheme 1 below:

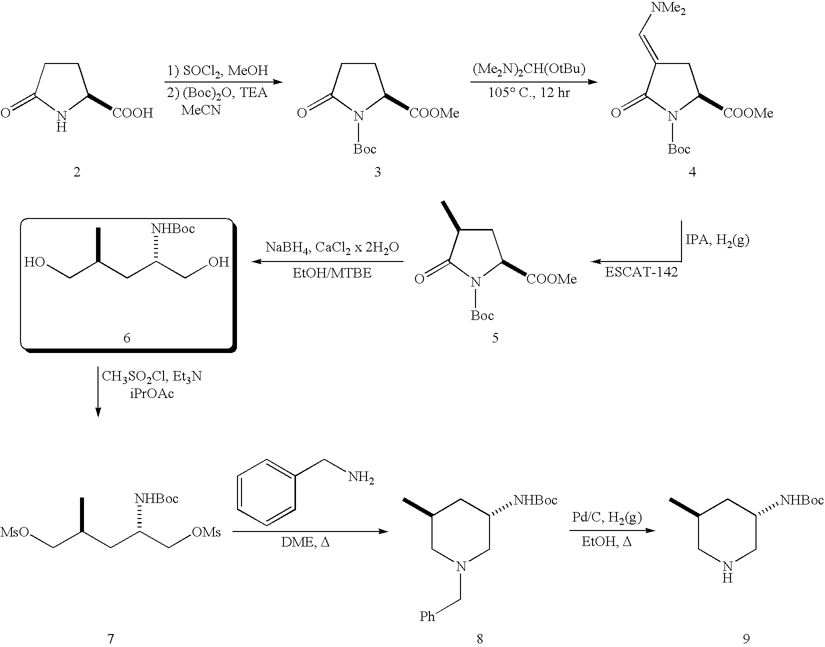

A 50-L reactor was charged with Compound 2 (5.50 kg, 42.60 mol), methanol (27 L) and cooled to 10-15° C. Thionyl chloride (10.11 kg, 2.0 equiv.) was added via an addition funnel over a period of 65 min, with external cooling to keep temperature below 30°. The resulting solution was stirred at 25° C. for 1.0 hour, after which methanol was removed under reduced pressure. The oily residue was azeotroped with ethyl acetate (3×2.5 L) to remove residual methanol, dissolved in ethyl acetate (27.4 L), charged into a 50 L reactor, and neutralized by slow addition of triethylamine (3.6 kg) below 30° C. The resulting suspension was filtered to remove triethylamine hydrochloride.

The filtrate was charged to a 50 L reactor, along with DMAP (0.53 kg). Di-tert-butyl dicarbonate (8.43 kg) was added via hot water heated addition funnel, over a period of 30 min at a temperature of 20-30° C. The reaction was complete after 1 hour as determined by TLC analysis. The organic phase was washed with ice cold 1N HCl (2×7.5 L), saturated sodium bicarbonate solution (1×7.5 L), dried over magnesium sulfate, and filtered. After ethyl acetate was removed under reduced pressure, crystalline slurry was obtained, triturated with MTBE (10.0 L), and filtered to afford Compound 3 as a white solid (5.45 kg, 52.4%).

Anal. Calcd for C₁₁H₁₇NO₅: C, 54.3; H, 7.04; N, 5.76. Found: C, 54.5; H, 6.96; N, 5.80. HRMS (ESI⁺) Expected for C₁₁H₁₈NO₅, [M+H] 244.1185. Found 244.1174; ¹H NMR (CDCl₃, 500 MHz):δ=4.54 (dd, J=3.1, 9.5 Hz, 1H), 3.7 (s, 3H), 2.58-2.50 (m, 1H), 2.41 (ddd, 1H, J=17.6, 9.5, 3.7), 2.30-2.23 (m, 1H), 1.98-1.93 (m, 1H), 1.40 (s, 9H); ¹³C NMR (CDCl₃, 125.70 MHz) 6 173.3, 171.9, 149.2, 83.5, 58.8, 52.5, 31.1, 27.9, 21.5; Mp 70.2° C.

A 50-L reactor was charged with Compound 3 (7.25 kg, 28.8 mol), DME (6.31 kg), and Bredereck's Reagent (7.7 kg, 44.2 mole). The solution was agitated and heated to 75° C.±5° C. for three hours. The reaction was cooled to 0° C. over an hour, during which time a precipitate formed. The mixture was kept at 0° C. for an hour, filtered, and dried in a vacuum oven for at least 30 hours at 30° C.±5° C. to give compound 4 as a white crystalline solid (6.93 kg, 77.9%).

Anal. Calcd for C₁₄H₂₂N₂O₅: C, 56.4; H, 7.43; N, 9.39. Found C, 56.4; H, 7.32; N, 9.48; HRMS (ESI⁺) Expected for C₁₄H₂₂N₂O₅, [M+H] 299.1607. Found 299.1613; ¹H NMR (CDCl₃, 499.8 MHz) δ=7.11 (s, 1H), 4.54 (dd, 1H, J=10.8, 3.6), 3.74 (s, 3H), 3.28-3.19 (m, 1H), 3.00 (s, 6H), 2.97-2.85 (m,1H), 1.48 (s, 9H); ¹³C NMR (CDCl₃, 125.7 MHz) δ=172.6, 169.5, 150.5, 146.5, 90.8, 82.2, 56.0, 52.3, 42.0, 28.1, 26.3. MP 127.9° C.

A 10-gallon Pfaudler reactor was charged with ESCAT 142 (Engelhard Corp. N.J, US) 5% palladium powder on carbon (50% wet, 0.58 kg wet wt.), Compound 4 (1.89 kg, 6.33 mol), and isopropanol (22.4 Kg). After agitated under a 45-psi hydrogen atmosphere at 45° C. for 18 hrs, the reaction mixture was cooled to room temperature and filtered though a bed of Celite (0.51 kg). The filtrate was evaporated under reduced pressure to give a thick oil, which was solidified on standing to afford Compound 5 (1.69 kg, 100%) as a 93:7 diastereomeric mixture.

A sample of product mixture was purified by preparative HPLC to give material for analytical data. Anal. Calcd for C₁₂H₁₉NO₅: C, 56.0; H, 7.44; N, 5.44. Found C, 55.8; H, 7.31; N, 5.44; MS (ESI⁺) Expected for C₁₂H₁₉NO₅, [M+H] 258.1342. Found 258.1321; ¹H NMR (CDCl₃, 499.8 MHz) δ=4.44 (m, 1H), 3.72 (s, 3H), 2.60-2.48 (m, 2H), 1.59-1.54 (m, 1H), 1.43 (s, 9H), 1.20 (d, j=6.8 Hz,3H); ¹³C NMR (CDCl₃, 125.7 MHz) δ=175.7, 172.1, 149.5, 83.6, 57.4, 52.5, 37.5, 29.8, 27.9, 16.2. Mp 89.9° C.

A 50-L reactor was charged with Compound 5 (3.02 kg, 11.7 mol), absolute ethanol (8.22 kg), and MTBE (14.81 kg). Sodium borohydride (1.36 kg, 35.9 mol) was added in small portions at 0° C.±5° C. A small amount of effervescence was observed. The reaction mixture was warmed to 10° C.±5° C. and calcium chloride dihydrate (2.65 kg) was added in portions at 10° C.±5° C. over an hour. The reaction was allowed to warm to 20° C.±5° C. over one hour and agitated for an additional 12 hours at 20° C.±5° C. After the reaction was cooled to −5° C.±5° C., ice-cold 2N HCl (26.9 kg) was added slowly at of 0° C.±5° C. Agitation was stopped. The lower aqueous phase was removed. The reactor was charged with aqueous saturated sodium bicarbonate (15.6 kg) over five minutes under agitation. Agitation was stopped again and the lower aqueous phase was removed. The reactor was charged with magnesium sulfate (2.5 kg) and agitated for at least 10 minutes. The mixture was filtered though a nutsche filter, and concentrated under reduced pressure to afford Compound 6 (1.80 kg, 66%).

Anal. Calcd for C₁₁H₂₃NO₄: C, 56.6 H, 9.94; N, 6.00. Found C, 56.0; H, 9.68; N, 5.96; HRMS (ESI⁺) Expected for C₁₁H₂₄NO₄, [M+H] 234.1705. Found 234.1703; ¹H NMR (CDCl₃, 500 MHz) δ=6.34 (d, J=8.9 Hz, 1H, NH), 4.51 (t, J=5.8, 5.3 Hz, 1H, NHCHCH₂OH), 4.34 (t, J=5.3, 5.3 Hz, 1H, CH3CHCH₂OH), 3.46-3.45, (m, 1H, NHCH), 3.28 (dd, J=10.6, 5.3 Hz, NHCHCHHOH), 3.21 (dd, J=10.2, 5.8 Hz, 1H, CH₃CHCHHOH), 3.16 (dd, J=10.2, 6.2 Hz, 1H, NHCHCHHOH), 3.12 (dd, J=10.6, 7.1 Hz, 1H, CH₃CHCHHOH), 1.53-1.50 (m, 1H, CH₃CHCHHOH), 1.35 (s, 9H, O(CH₃)₃, 1.30 (ddd, J=13.9, 10.2, 3.7 Hz, 1H, NHCHCHHCH), 1.14 (ddd, J=13.6, 10.2, 3.4 Hz, 1H, NHCHCHHCH), 0.80 (d, J=6.6 Hz, 3H, CH₃); ¹³C NMR (CDCl₃, 125.7 MHz) δ 156.1, 77.9, 50.8, 65.1, 67.6, 65.1, 35.6, 32.8, 29.0, 17.1. Mp 92.1° C.

A 50 L reactor was charged with a solution of Compound 6 (5.1 kg) in isopropyl acetate (19.7 kg). The reaction was cooled to 15° C.±5° C. and triethylamine (7.8 kg) was added at that temperature. The reactor was further cooled to 0° C.±5° C. and methanesulfonyl chloride (MsCl) (6.6 kg) was added. The reaction was stirred for a few hours and monitored for completion by HPLC or TLC. The reaction was quenched by saturated aqueous bicarbonate solution. The organic phase was isolated and washed successively with cold 10% aqueous triethylamine solution, cold aqueous HCl solution, cold saturated aqueous bicarbonate solution, and finally saturated aqueous brine solution. The organic phase was dried, filtered, and concentrated in vacuo below 55° C.±5° C. to afford compound 7 as a solid/liquid slurry, which was used in the subsequent reaction without further purification.

After charged with 9.1 kg of neat benzylamine, a 50 L reactor was warmed to 55° C., at which temperature, a solution of compound 7 (8.2 kg) in 1,2-dimethoxyethane (14.1 kg) was added. After the addition, the reaction was stirred at 60° C.±5° C. for several hours and monitored for completion by TLC or HPLC. The reaction was cooled to ambient temperature and the solvent was removed under vacuum. The residue was diluted with 11.7 kg of 15% (v/v) ethyl acetate/hexanes solution and treated, while agitating, with 18.7 kg of 20% (wt) aqueous potassium carbonate solution. A triphasic mixture was obtained upon standing. The upper organic layer was collected. The isolated middle layer was extracted twice again with 11.7 kg portions of 15% (v/v) ethyl acetate/hexanes solution. The combined organic layers were concentrated under vacuum to give an oily residue. The residue was then purified by chromatography to afford Compound 8 as an oil.

A 40 L pressure vessel was charged with 0.6 kg 50% wet, solid palladium on carbon (E101, 10 wt. %) under flow of nitrogen. A solution of Compound 8 (3.2 kg) in 13.7 kg of absolute ethanol was then added to the reactor under nitrogen. The reactor was purged with nitrogen and then pressurized with hydrogen at 45 psi. The reaction was then heated to 45° C. It was monitored by TLC or LC. Upon completion, the reaction was cooled to ambient temperature, vented, and purged with nitrogen. The mixture was filtered through a bed of Celite and the solid was washed with 2.8 kg of absolute ethanol. The filtrate was concentrated under vacuum to afford Compound 9 as a waxy solid.

TLC R_{f} (Silica F₂₅₄, 70:30 v/v ethyl acetate-hexanes, KMnO₄ stain)=0.12; ¹H NMR (300 MHz, CDCl₃) δ 5.31 (br s, 1H), 3.80-3.68 (m, 1H), 2.92 (d, J=11.4 Hz, 1H), 2.77 (AB quart, J_{AB}=12.0 Hz, ν=50.2 Hz, 2H), 2.19 (t, J=10.7 Hz, 1H), 1.82-1.68 (m, 2H), 1.54 (br s, 1H), 1.43 (s, 9H), 1.25-1.15 (m, 1H), 0.83 (d, J=6.6 Hz, 3H); ¹³C NMR (75 MHz, CDCl₃) δ: 155.3, 78.9, 54.3, 50.8, 45.3, 37.9, 28.4, 27.1, 19.2; MS (ESI+) m/z 215 (M+H), 429 (2M+H).

Similarly, (3S,5R)-(5-Methyl-piperidin-3-yl)-carbamic acid tert-butyl ester (Compound 9′) was synthesized as shown in Scheme 2.

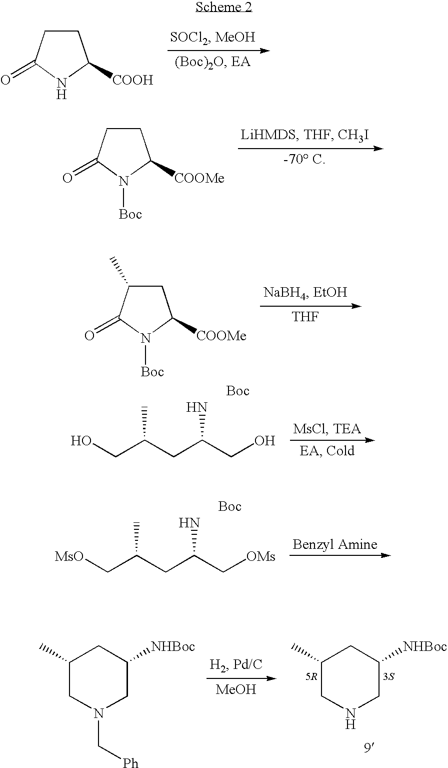

### (B) Synthesis of 1-Cyclopropyl-7-fluoro-8-methoxy-4-oxo-1,4-dihydro-quinoline-3-carboxylic acid (Compound 10)

Compound 10 was prepared according to the method described in U.S. Pat. No. 6,329,391.

### (C) Synthesis of borone ester chelate of 1-Cyclopropyl-7-fluoro-8-methoxy-4-oxo-1,4-dihydro-quinoline-3-carboxylic acid (Compound 11)

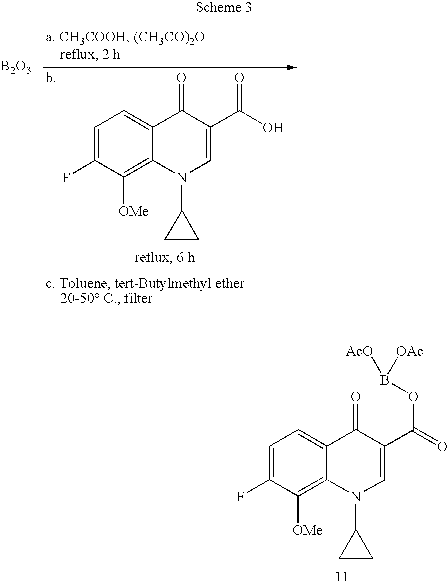

A reactor was charged with boron oxide (2.0 kg, 29 mol), glacial acetic acid (8.1 L, 142 mol), and acetic anhydride (16.2 L, 171 mol). The resulting mixture was refluxed at least 2 hours, and then cooled to 40° C., at which temperature, 7-fluoroquinolone acid compound 10 (14.2 kg, 51 mol) was added. The mixture was refluxed for at least 6 hours, and then cooled to about 90° C. Toluene (45 L) was added to the reaction. At 50° C., tert-butylmethyl ether (19 L) was added to introduce precipitation. The mixture was then cooled to 20° C. and filtered to isolate the precipitation. The isolated solid was then washed with tert-butylmethyl ether (26 L) prior to drying in a vacuum oven at 40° C. (50 torr) to afford Compound 11 in a yield of 86.4%.

Raman (cm⁻¹): 3084.7, 3022.3, 2930.8, 1709.2, 1620.8, 1548.5, 1468.0, 1397.7, 1368.3, 1338.5, 1201.5, 955.3, 653.9, 580.7, 552.8, 384.0, 305.8. NMR (CDCl₃, 300 MHz) δ (ppm): 9.22 (s, 1H), 8.38-8.33 (m, 1H), 7.54 (t, J=9.8 Hz, 1H), 4.38-4.35 (m, 1H), 4.13 (s, 3H), 2.04 (s, 6H), 1.42-1.38 (m, 2H), 1.34-1.29 (m, 2H). TLC (Whatman MKC18F Silica, 60 Å, 200 μm), Mobile Phase: 1:1 (v/v) CH₃CN: 0.5N NaCl (aq), UV (254/366 nm) visualization; R_{f}=0.4-0.5.

### (D) Synthesis of malate salt hemihydrate of (3S,5S)-7-[3-amino-5-methyl-piperidinyl]-1-cyclopropyl-1,4-dihydro-8-methoxy-4-oxo-3-quinolinecarboxylic acid (Compound 1) and malate salt hemihydrate of (3S,5R)-7-[3-amino-5-methyl-piperidinyl]-1-cyclopropyl-1,4-dihydro-8-methoxy-4-oxo-3-quinolinecarboxylic acid (Compound 1′)

Compound 1 was synthesized from compound 9 as shown in Scheme 4 below:

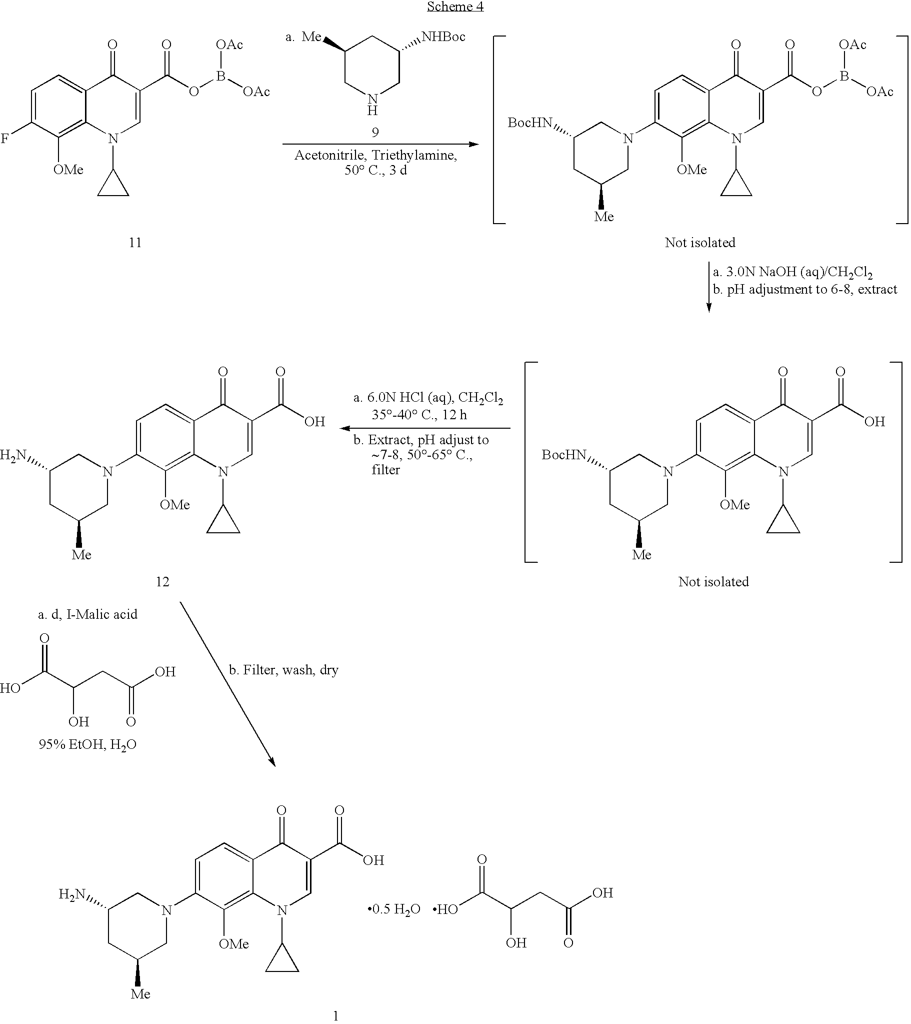

A reactor was charged with Compound 11 (4.4 kg, 10.9 mol), Compound 9 (2.1 kg, 9.8 mol), triethylamine (TEA) (2.1 L, 14.8 mol), and acetonitrile (33.5 L, 15.7 L/kg). The resulting mixture was stirred at approximately 50° C. till completion of the reaction, as monitored by HPLC or reverse phase TLC. It was cooled to approximately 35° C. and the reaction volume was reduced to approximately half by distillation of acetonitrile under vacuum between 0-400 torr. After 28.2 kg of 3.0 N NaOH (aq) solution was added, the reaction mixture was warmed to approximately 40° C., distilled under vacuum until no further distillates were observed, and hydrolyzed at room temperature. Upon completion of hydrolysis, which was monitored by HPLC or reverse phase TLC, 4-5 kg of glacial acetic acid was added to neutralize the reaction mixture.

The resulting solution was extracted 3 times with 12.7 kg (9.6 L) of dichloromethane. The organic layers were combined and transferred to another reactor. The reaction volume was reduced to approximately an half by evaporation at 40° C. After 20.2 Kg 6.0N HCl (aq) solution was added, the reaction mixture was stirred for at least 12 hours at 35° C. After the reaction was completed as monitored by HPLC or reverse phase TLC, agitation was discontinued to allow phase separation. The organic phase was removed and the aqueous layer was extracted with 12.7 kg (9.6 L) of dichloromethane. The aqueous layer was diluted with 18.3 kg distilled water and warmed to approximately 50° C. Dichloromethane was further removed by distillation under vacuum (100-400 torr).

The pH of the aqueous solution was then adjusted to 7.8-8.1 by adding about 9.42 kg of 3.0 N NaOH (aq) below 65° C. The reaction mixture was stirred at 50° C. for at least an hour and then cooled to room temperature. The precipitate was isolated by suction filtration, washed twice with 5.2 kg of distilled water, and dried with suction for at least 12 hours and then in a convection oven at 55° C. for additional 12 hours. Compound 12 (3.2 kg, 79%) was obtained as a solid.

A reactor was charged with 3.2 kg of Compound 12 and 25.6 kg of 95% ethanol. To the reactor was added 1.1 kg of solid D,L-malic acid. The mixture was refluxed temperature (˜80° C.). Distilled water (˜5.7 L) was added to dissolve the precipice and 0.2 kg of activated charcoal was added. The reaction mixture was passed through a filter. The clear filtrate was cooled to 45° C. and allowed to sit for at least 2 hours to allow crystallization. After the reaction mixture was further cooled to 5° C., the precipitate was isolated by suction filtration, washed with 6.6 kg of 95% ethanol, and dried with suction for at least 4 hours. The solid was further dried in a convection oven at 45° C. for at least 12 hours to afford 3.1 kg of Compound 1 (yield: 70%).

NMR (D₂O, 300 MHz) δ (ppm): 8.54 (s, 1H), 7.37 (d, J=9.0 Hz, 1H), 7.05 (d, J=9.0 Hz, 1H), 4.23-4.18 (m, 1H), 4.10-3.89 (m, 1H), 3.66 (br s, 1H), 3.58 (s, 3H), 3.45 (d, J=9.0 Hz, 1H), 3.34 (d, J=9.3 Hz, 1H), 3.16 (d, J=12.9 Hz, 1H), 2.65 (dd, J=16.1, 4.1 Hz, 1H), 2.64-2.53 (m, 1H), 2.46 (dd, J=16.1, 8.0 Hz, 1H), 2.06 (br s, 1H), 1.87 (d, J=14.4 Hz, 1H), 1.58-1.45 (m, 1H), 1.15-0.95 (m, 2H), 0.91 (d, J=6.3 Hz, 3H), 0.85-0.78 (m, 2H).

Similarly, Compound 1′ was synthesized from Compound 9′ as shown in Scheme 5 below:

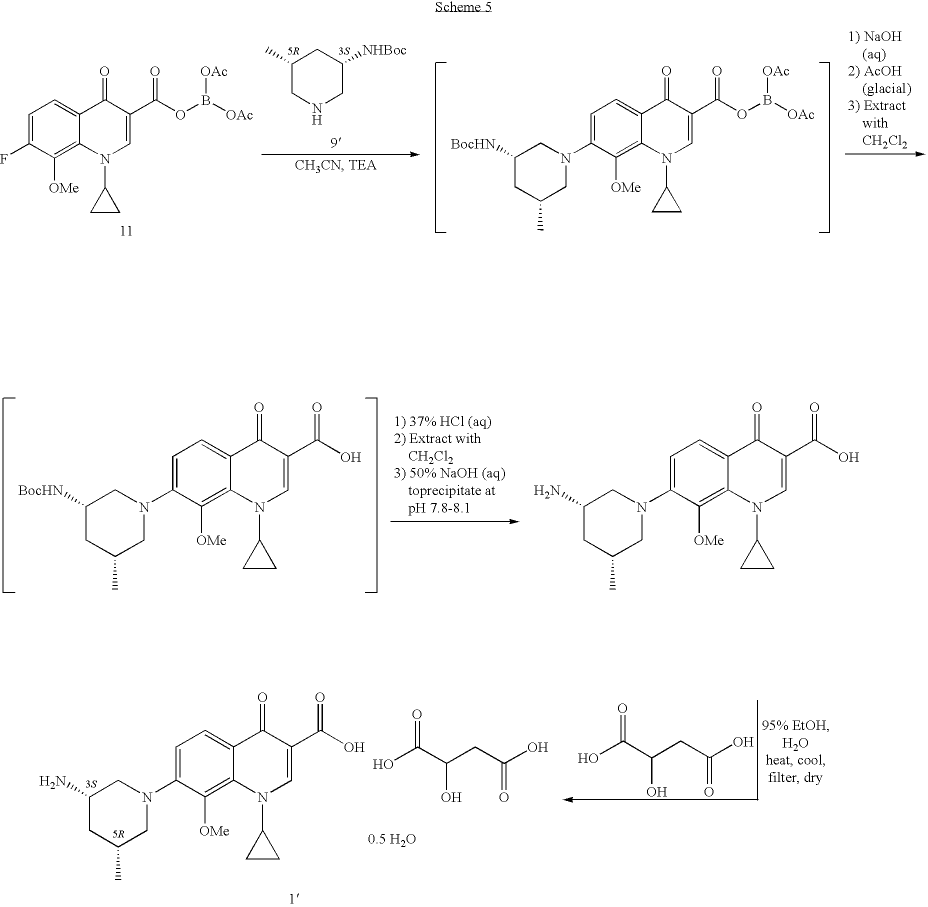

### Example 2

Compound 1-containing capsules and levofloxacin capsules were prepared as follows:

Compound 1 (malate salt hemihydrate of (3S,5S)-7-[3-amino-5-methyl-piperidinyl]-1-cyclopropyl-1,4-dihydro-8-methoxy-4-oxo-3-quinolinecarboxylic acid), microcrystalline cellulose, and magnesium stearate were mixed at a ratio of 119:33.5:1. 445.0 mg of the mixture was enclosed in a gelatin capsule shell (blue cap and blue body, size 0) to afford a drug capsule.

| Component | Unit Quantity (mg/capsule) |
|---|---|
| Compound 1 | 345.0* |
| microcrystalline cellulose, USP/NF/EP | 97.1 |
| magnesium stearate, USP/NF/EP | 2.9 |
| Total Fill Weight | 445.0 |

*equivalent to 250 mg of the free base compound, i.e., (3S,5S)-7-[3-amino-5-methyl-piperidinyl]-1-cyclopropyl-1,4-dihydro-8-methoxy-4-oxo-3-quinolinecarboxylic acid.

Each of levofloxacin capsules was prepared by enclosing in a gelatin capsule shell (commercially available) a 250 mg levofloxacin tablet (also commercially available) and approximately 50 mg of microcrystalline cellulose.

### Example 3

Randomized, double-blind clinical trials were conducted at 17 sites in Taiwan and South Africa to assess the effectiveness of Compound 1 in treating adult patients with community-acquired pneumonia.

The trials included a total of 265 subjects having community-acquired pneumonia. The average age of the subjects was 43.5 years old and the average body weight was 66.17 kg. About 50% of the subjects were male, and about 62% of the subjects were Black or African American, about 21% White, and about 15% Asian.

Among the 265 subjects, 86 were treated with 3 Compound 1-containing capsules (750 mg of the free base compound, i.e., (3S,5S)-7-[3-amino-5-methyl-piperidinyl]-1-cyclopropyl-1,4-dihydro-8-methoxy-4-oxo-3-quinolinecarboxylic acid) per day, 89 with 2 Compound 1-containing capsules (500 mg of the free base compound) per day, and 90 with 2 levofloxacin capsules (500 mg of levofloxacin) per day for 7 consecutive days. In each group, the drug capsules were orally taken by the subjects in the morning with a full glass of water (240 mL). Within 2 hours after taking the drugs, the subjects were not allowed to eat, but water intake (no more than 240 mL) was permitted. Overall, 10.6% of randomized subjects withdrew from the treatments.

The results show that, like leveoflooxacin, Compound 1 was effective in treating community-acquired pneumonia. More specifically, after 7 days, 71 subjects taking 750 mg of quinolone compound per day were cured (cure rate: 82.6%), 67 subjects taking 500 mg of quinolone per day were cured (cure rate: 75.3% ), and 72 subjects taking 500 mg levofloxacin per day were cured (cure rate: 80.0%).

### Example 4

Pharmacokinetic analysis was conducted to assess the safety of Compound 1.

Blood samples were collected from each subject taking Compound 1 on day 10 at 0 hour (pre-dose) and 0.5, 1, 1.5, 2, 4, 6, 8, 12, 16 and 24 hours (post-dose). 5 mL of each sample was transferred to a heparin sodium tube and immediately placed on ice. Plasma was separated by centrifugation at approximately 4° C. and transferred to appropriately labeled polypropylene specimen containers (two tubes with 1-1.5 mL plasma/tube) and frozen at approximately −70° C. before use.

Prior to analysis of the blood samples, pharmacokinetic assays were validated. The details of the assay validation are listed in the table below.

| | | | Accuracy | Precision |
|---|---|---|---|---|
| Analyte | Assay Type | LLOQ | (% of bias) | (% CV) |
| Compound 1 | in plasma | 5.0 ng/mL | −1.8~2.2% | 4.3~7.5% |

LLOQ: lower limit of quantitation (LLOQ)
CV: coefficient of variation (CV)

The pharmacokinetic assays of the blood samples were performed by Charles River Laboratories (Worcester, Mass.). Cₘₐₓ (Peak concentration of Compound 1 in plasma) and AUC₀₋₂₄ₕ (Area under the plasma concentration-time curve from 0 to 24 hours post-dosing, calculated by linear/log trapezoidal method) were determined from the plasma concentration-time data using non-compartmental approaches (WinNonlin version 4. 1, Pharsight Corporation, CA).

Protein binding was also measured as follows: Ultrafiltrate (UF) samples were obtained by centrifuging the above-mentioned Compound 1-contaning heparinized human plasma in molecular weight cutoff ultrafiltration devices (30,000 Da) at ˜3000 rpm (30 min, ˜37° C.). The UF samples (0.025 mL) were mixed with an internal standard solution—0.050 mL of ˜800 ng/mL O¹³CD₃-Compound 1 (in which the OCH₃ group was replaced with the O¹³CD₃ group), diluted by 20 folds, and analyzed by reverse-phase HPLC on a 3.5 micron C-18 column. Quantitation was obtained by the multiple reaction monitoring method through positive ion Turbo-Ion Spray ionization. Ultrafiltrate standards were used to quantify the unbound drug in plasma quality control samples and unknown specimens. Non-specific protein binding (NSB) was measured (NSB=0.0415) and used as a correction factor to determine the final % protein bindings. The nominal range of quantitation for the analyte was 50 to 10,000 ng/mL. 0.400 mL aliquot of human plasma was used in the assay. Sample concentrations were determined by back-calculation using a weighted linear (1/x²) regression of a calibration curve generated from spiked UF standards. Over the linear range, the inter-batch % CV for Compound 1 was 4.9% to 11.8%.

Shown in the table below are the AUC₀₋₂₄, Cₘₐₓ, and protein binding values of Compound 1 when the subjects took 500 mg, 750 mg, and 1000 mg per day. The free Cₘₐₓ and free AUC₀₋₂₄ values shown in the table are those that have been corrected for plasma protein binding. Also shown in the table are the ratios of free Cₘₐₓ/MIC and free AUC/MIC which are useful for prediction of clinical and microbiological outcome as well as bacterial resistance development. Free Cₘₐₓ/MIC greater than about 8 and free AUC/MIC greater than about 100 are preferred for antibiotic drugs.

| | | | | | Free AUC0-24/MIC90 ratios at steady state | | | | |
|---|---|---|---|---|---|---|---|---|---|
| | | AUC₀₋₂₄ | Protein | Free AUC₀₋₂₄ | MIC90 (μg/mL) | | | | |
| Antibiotic | Regimen | (hr*μg/mL) | Binding (%) | (hr*μg/mL) | 0.125 | 0.25 | 0.5 | 0.75 | 1 |
| Compound 1 | 500 mg q24 p.o. | 38.6 | 16 | 32.4 | 259 | 130 | 65 | 43 | 32 |
| | 750 mg q24 p.o. | 58.4 | 16 | 49.1 | 393 | 196 | 98 | 65 | 49 |
| | 1000 mg q24 p.o. | 74.8 | 16 | 62.9 | 503 | 251 | 126 | 84 | 63 |

| | | | | | Free Cmax/MIC90 ratios at steady state | | | | |
|---|---|---|---|---|---|---|---|---|---|
| | | Cₘₐₓ | Protein | Free Cₘₐₓ | MIC90 (μg/mL) | | | | |
| Antibiotic | Regimen | (μg/mL) | Binding (%) | (μg/mL) | 0.125 | 0.25 | 0.5 | 0.75 | 1 |
| Compound 1 | 500 mg q24 p.o. | 5.56 | 16 | 4.7 | 37 | 19 | 9 | 6 | 5 |
| | 750 mg q24 p.o. | 6.82 | 16 | 5.7 | 46 | 23 | 11 | 8 | 6 |
| | 1000 mg q24 p.o. | 8.20 | 16 | 6.9 | 55 | 28 | 14 | 9 | 7 |

### Example 5

In vitro assays were conducted to assess the effective of Compound 1 and Compound 1′ in inhibiting bacteria

Inhibition of Methicillin-Resistant Staphylococcus aureus by Compound 1

MRSA isolates (n=193) were obtained as part of the Canadian National Intensive Care Unit (CAN-ICU) Study. 19 medical centers from all regions of Canada with active ICUs participated in the CAN-ICU study included. They were requested to only obtain “clinically significant” specimens from patients with a presumed infectious disease. Surveillance swabs, eye, ear, nose and throat swabs were excluded. Anaerobic organisms and fungal organisms were also excluded.

From September 2005-June 2006 (inclusive), each center collected a maximum of 300 consecutive pathogens isolated from blood, urine, tissue/wound, and respiratory specimens (one pathogen per cultured site per patient) of ICU patients. These isolates were shipped to the reference laboratory (Health Sciences Centre, Winnipeg, Canada) on Amies charcoal swabs, subcultured in appropriate media, and stocked in skim milk at −80° C.

The isolates' methicillin resistance was confirmed using the disk diffusion method described by the Clinical and Laboratory Standards Institute. All isolates underwent mecA PCR, as well as molecular characterization (including PVL analysis and fingerprinting), as previously described, to assess whether they were community-associated or healthcare-associated (Christianson et al., J Clin Microbiol. 2007, 45 (6): 1904-11; Mulvey et al., J Clin. Microbiol. 2001, 39(10): 3481-5; Mulvey et al., Emerg Infect Dis. 2005,11(6): 844-50; Oliveira et al., Antimicrob Agents Chemother. 2002, 46(7): 2155-61). The isolates were also subtyped using pulsed-field gel electrophoresis (PFGE) following the Canadian standardized protocol as previously described (Mulvey et al., J Clin Microbiol. 2001, 39(10): 3481-5). Their PFGE fingerprints thus obtained were analyzed with BioNumerics v3.5 (Applied Maths St. Marten-Latem, Belgium) using a position tolerance of 1.0 and an optimization of 1.0. Strain relatedness was determined as previously described (Tenover et al., 1995). The fingerprints of the isolates were compared to the national MRSA fingerprint database and were grouped into one of 10 Canadian epidemic MRSA (CMRSA-1, CMRSA-2, etc) as previously described (Mulvey et al., Emerg Infect Dis. 2005,11(6):844-50). The MRSA isolates belong to genotypes: CMRSA-1 (USA600), CMRSA-2 (USA 100), CMRSA-4 (USA200), CMRSA-7 (USA400, MW2) and CMRSA-10 (USA300). USA 300 and USA 400 are community-associated MRSA (CA-MRSA) strains and USA 200 and USA 600 are healthcare-associated MRSA strains.

Compound 1 and other antibiotics were tested for their inhibitory activity against the MRSA isolates using the broth microdilution guidelines as stipulated by the Clinical and Laboratory Standards Institute. The results show that Compound 1 effectively inhibited MRSA. It was also found that this compound was more active against community-associated-MRSA strains than healthcare-associated-MRSA strains.

### Inhibition of Multidrug-Resistant Methicillin-Resistant Staphylococcus Aureus by Compound 1

Compound 1 was tested for its inhibitory effect against multidrug-resistant methicillin-resistant Staphylococcus aureus obtained by 10 medical centers in all regions of Taiwan. MICs were determined using the agar dilution methods recommended by the Clinical and Laboratory Standards Institute (CLSI-M100-S18).

The results show that Compound 1 was effective in inhibiting the MRSA isolates that are Ciprofloxacin-resistant, Vancomycin-intermediate-resistant, and Daptomycin-nonsusceptible.

### Inhibition of Antibiotic-Resistant Bacterial by Compound 1′

Compound 1′, Ciprofloxacin, and Levofloxacin were tested for their inhibitory effect against methicillin-resistant Staphylococcus aureus and methicillin-resistant Streptococcus pneumoniae at various concentrations between 0.008 and 8 μg/ml on 10 different days. The Staphylococcus aureus and Streptococcus pneumoniae isolates were obtained by 10 medical centers in all regions of Taiwan. MICs were determined using the broth microdilution method.

The results show that Compound 1′ was effective in inhibiting methicillin-resistant Staphylococcus aureus and Streptococcus pneumoniae.

### Other Embodiments

All of the features disclosed in this specification may be combined in any combination. An alternative feature serving the same, equivalent, or similar purpose may replace each feature disclosed in this specification. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

What is claimed is:

1. A method of treating pneumonia comprising orally administering to a subject in need thereof a composition containing a compound of the following formula: 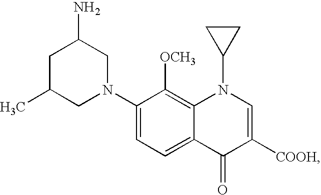
at a daily dose of 2-30 mg/kg.

2. The method of claim 1, wherein the compound is in the salt form.

3. The method of claim 2, wherein the compound is in the malic acid salt form.

4. The method of claim 3, wherein the compound is in the malic acid salt hemihydrate form.

5. The method of claim 4, wherein the composition further contains microcrystalline cellulose and magnesium stearate and is in the form of a capsule or tablet.

6. The method of claim 5, wherein the pneumonia is caused by methicillin-, vancomycin-, or penicillin-nonsusceptible bacteria, the bacteria being Streptococcus pneumoniae, Haemophilus influenzae, Mycoplasma pneumoniae, Legionella pneumophila, Moraxella catarrhalis, Mycobacterium tuberculosis, or Chlamydophilia pneumoniae.

7. The method of claim 6, wherein the daily dose is 7-12 mg/kg.

8. The method of claim 1, wherein the compound is

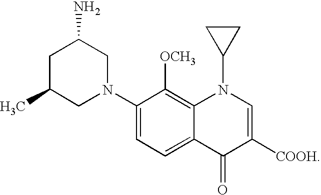

9. The method of claim 8, wherein the compound is in the malic acid salt hemihydrate form.

10. The method of claim 9, wherein the composition further contains microcrystalline cellulose and magnesium stearate and is in the form of a capsule or tablet.

11. The method of claim 10, wherein the pneumonia is caused by methicillin-, vancomycin-, or penicillin-nonsusceptible bacteria, the bacteria being Streptococcus pneumoniae, Haemophilus influenzae, Mycoplasma pneumoniae, Legionella pneumophila, Moraxella catarrhalis, Mycobacterium tuberculosis, or Chlamydophilia pneumoniae.

12. The method of claim 6, wherein the daily dose is 7-12 mg/kg.

13. The method of claim 1, wherein the compound is

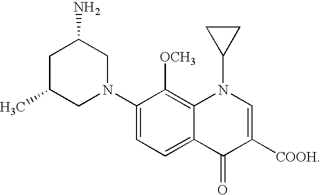

14. The method of claim 13, wherein the compound is in the malic acid salt hemihydrate form.

15. The method of claim 14, wherein the composition further contains microcrystalline cellulose and magnesium stearate and is in the form of a capsule or tablet.

16. The method of claim 15, wherein the pneumonia is caused by methicillin-, vancomycin-, or penicillin-nonsusceptible bacteria, the bacteria being Streptococcus pneumoniae, Haemophilus influenzae, Mycoplasma pneumoniae, Legionella pneumophila, Moraxella catarrhalis, Mycobacterium tuberculosis, or Chlamydophilia pneumoniae.

17. The method of claim 16, wherein the daily dose is 7-12 mg/kg.

18. The method of claim 1, wherein the composition further contains microcrystalline cellulose and magnesium stearate and is in the form of a capsule or tablet.

19. The method of claim 1, wherein the pneumonia is caused by methicillin-, vancomycin-, or penicillin-nonsusceptible bacteria, the bacteria being Streptococcus pneumoniae, Haemophilus influenzae, Mycoplasma pneumoniae, Legionella pneumophila, Moraxella catarrhalis, Mycobacterium tuberculosis, or Chlamydophilia pneumoniae.

20. The method of claim 1, wherein the daily dose is 7-12 mg/kg.

21. The method of claim 1, wherein the pneumonia is community-acquired pneumonia.

22. The method of claim 8, wherein the pneumonia is community-acquired pneumonia.

23. The method of claim 13, wherein the pneumonia is community-acquired pneumonia.

24. The method of claim 1, wherein the daily dose of the compound is 3-16 mg/kg.

25. The method of claim 24, wherein the compound is in the malic acid salt hemihydrate form.

26. The method of claim 25, wherein the composition further contains microcrystalline cellulose and magnesium stearate and is in the form of a capsule or tablet.

27. The method of claim 26, wherein the pneumonia is caused by methicillin-, vancomycin-, or penicillin-nonsusceptible bacteria, the bacteria being Streptococcus pneumoniae, Haemophilus influenzae, Mycoplasma pneumoniae, Legionella pneumophila, Moraxella catarrhalis, Mycobacterium tuberculosis, or Chlamydophilia pneumoniae.
